(19) United States
(12) Patent Application Publication
(10) Pub. No.: US 2026/0049156 A1
(43) Pub. Date: Feb. 19, 2026

(54) ANTI-TYRP1 BI-SPECIFIC T CELL ENGAGING PROTEIN FOR TREATMENT OF TYRP1-EXPRESSING MELANOMA

(71) Applicant: Northwestern University, Evanston, IL (US)

(72) Inventors: Irina V. Balyasnikova, Evanston, IL (US); Isabelle Caroline Le Poole, Evanston, IL (US)

(21) Appl. No.: 19/103,495

(22) PCT Filed: Aug. 16, 2023
(86) PCT No.: PCT/US2023/072328

§ 371 Date: Feb. 12, 2025

Related U.S. Application Data
(60) Provisional application No. 63/398,435, filed on Aug. 16, 2022.

(51) Int. Cl. C07K 16/40; A61K 35/17; A61K 39/00; A61K 40/11; A61K 40/33; A61P 35/00; C07K 16/28

(52) U.S. Cl. CPC C07K 16/40; A61K 35/17; A61K 40/11; A61K 40/33; A61P 35/00; C07K 16/2809; A61K 2039/505; C07K 2317/31; C07K 2317/565; C07K 2317/622; C07K 2319/02; C07K 2319/21

(57) ABSTRACT

The present invention provides bispecific antibodies that target T cells to melanoma cells. The bispecific antibodies comprise a first single-chain variable fragment (scFv) that binds to the T cell co-receptor CD3ε and a second scFv that binds to the melanoma marker protein tyrosinase-related protein 1 (TYRP1). Methods of using the bispecific antibodies to treat tumors and lyse target cells are also provided.

### CROSS-REFERENCE TO RELATED APPLICATIONS

This application claims priority to U.S. Provisional Application No. 63/398,435 filed on Aug. 16, 2022, the contents of which are incorporated by reference in their entireties.

### STATEMENT REGARDING FEDERALLY SPONSORED RESEARCH

NR

### SEQUENCE LISTING STATEMENT

This application includes a sequence listing in XML format titled “702581.02377_ST26.xml”, which is 29,399 bytes in size and was created on Aug. 15, 2023. The sequence listing is electronically submitted with this application via Patent Center and is incorporated herein by reference in its entirety.

### BACKGROUND

Melanoma is the most dangerous type of skin cancer, as it results in tumors that readily metastasize. Melanoma develops from pigment-producing cells known as melanocytes. Melanomas typically occur in the skin but also rarely occur in the mouth, intestines, or eye. About 25% of melanomas develop from moles. The primary cause of melanoma is ultraviolet light (UV) exposure in those with low levels of the skin pigment melanin. Diagnosis of melanoma is performed via biopsy and analysis of any skin lesion that has signs of being potentially cancerous.

Treatment of melanoma typically involves removal by surgery, which cures most patients with localized disease. However, in patients in which metastasis has occurred, the survival rate is only 65% when the disease has spread to lymph nodes, and 25% when distant spread has occurred. Accordingly, there remains a need in the art for improved methods for treating melanoma.

### SUMMARY

In a first aspect, the present invention provides bispecific antibodies (i.e., BTEs) that target T cells to melanoma cells and promote their activation. The bispecific antibodies comprise a first single-chain variable fragment (scFv) that binds to CD3ε and a second scFv that binds to tyrosinase-related protein 1 (TYRP1). The first scFv comprises: (a) a first heavy chain variable domain (V_{H}) comprising a CDR1 of SEQ ID NO:5, a CDR2 of SEQ ID NO:6, and a CDR3 of SEQ ID NO:7; (b) a first linker; and (c) a first light chain variable domain (V_{L}) comprising a CDR1 of SEQ ID NO:9, a CDR2 having the amino acid sequence DAS, and a CDR3 of SEQ ID NO:10. The second scFv comprises: (a) a second VL comprising a CDR1 of SEQ ID NO:13, a CDR2 having the amino acid sequence DAK, and a CDR3 of SEQ ID NO:14; (b) a second linker; and (c) a second VH comprising a CDR1 of SEQ ID NO:16, a CDR2 of SEQ ID NO:17, and a CDR3 of SEQ ID NO:18.

In a second aspect, the present invention provides pharmaceutical compositions comprising a bispecific antibody described herein and a pharmaceutically acceptable carrier.

In a third aspect, the present invention provides nucleic acids encoding a bispecific antibody described herein.

In a fourth aspect, the present invention provides vectors comprising a nucleic acid described herein.

In a fifth aspect, the present invention provides transgenic cells that express a bispecific antibody described herein.

In a sixth aspect, the present invention provides methods of treating a TYRP1-expressing tumor in a subject. The methods comprise administering a therapeutically effective amount of a bispecific antibody, pharmaceutical composition, or transgenic cell described herein to the subject to treat the tumor.

In a seventh aspect, the present invention provides methods for inducing lysis of a TYRP1-expressing target cell. The methods comprise contacting the target cell with a bispecific antibody described herein in the presence of a T cell in an amount effective to lyse the target cell.

### BRIEF DESCRIPTION OF THE DRAWINGS

FIG. 1 shows a western blot analysis of native and denatured forms of the tyrosinase-related protein 1 (TYRP1) bispecific T-cell engager (BTE). 0.5 μg protein was run in each lane. Detection was accomplished using an anti-6×His-HRP antibody (Abcam).

FIG. 2 compares TYRP1 surface expression in melanoma cells and melanocytes. Fluorescence-activated cell sorting (FACS) signal intensity of unfixed primary melanocytes (Mf0978), mouse melanoma cells (B16.F10), and human melanoma cells (624.38 and 888 A2) stained for TYRP1 surface expression using an anti-TYRP1 primary antibody (TA99) and a FITC-labeled goat-anti-mouse secondary antibody (+ primary antibody), compared to secondary controls that received no primary antibody (− primary antibody).

FIG. 3 demonstrates that the TYRP1 BTE promotes T-cell IFN-7 cytokine production in response to TYRP1-expressing melanoma cells. Healthy T cells purified from peripheral blood mononuclear cells (PBMC) were co-cultured at a 10:1 effector:target ratio with 888 A2 human melanoma cells, Mf22003 early passage (<5) normal human melanocytes, or negative control HEK293 cells, in the presence or absence of the TYRP1 BTE (1.25, 2.5, or 5 μg/ml). Supernatants were harvested after 48 hours, and IFN-γ concentrations were measured via sandwich enzyme-linked immunoassay (ELISA). This experiment was performed in triplicate.

FIG. 4 demonstrates that the TYRP1 BTE promotes T-cell cytotoxicity against TYRP1-expressing melanoma cells. T cells were co-cultured at a 10:1 effector:target ratio with 888 A2 human melanoma cells, Mf22003 human melanocytes, and negative control HEK293 cells in the presence or absence of the TYRP1 BTE (5 μg/ml). The viability of the target cells was measured every 3 hours via IncuCyte analysis, and the fold-change was calculated.

FIG. 5 demonstrates that the TYRP1 BTE promotes T cell production of inflammatory cytokines in response to TYRP1-expressing melanoma cells. T cells were co-cultured at a 10:1 effector:target ratio with 888 A2 human melanoma cells, Mf22003 human melanocytes, or negative control HEK293 cells, in the presence or absence of the TYRP1 BTE (1.25, 2.5, or 5 μg/ml). A bulk multiplex cytokine analysis was performed using the Mesoscale discovery platform. The concentrations of cytokines IL-1β, IL-6, IL-8, IL-4, IL-13, IL-2, TNF-α, IL-10, and IL-12p70 were measured.

FIGS. 6A-6B demonstrate that the TYRP1 BTE promotes T cell-mediated tumor growth suppression in vivo. Mice were subcutaneously injected with 888 A2 melanoma cells, and tumor size was monitored. Then, the mice were either treated with a combination of the TYRP1 BTE and T cells or left untreated. In FIG. 6A, individual tumor volumes are shown over time, and the arrows indicate treatment dates. In FIG. 6B, fold-change in tumor size is shown from the onset of treatment, and the arrow indicates the repeat treatment date.

FIGS. 7A-7B demonstrate that TYRP1 is expressed in melanoma brain metastases. A comparative analysis of TYRP1 mRNA levels in melanoma extracranial metastases and melanoma brain metastases was performed using the GSCE50493 data set. FIG. 7A shows TYRP1 expression in 5 paired samples of extracranial tissue and intracranial tissue derived from the same patient. FIG. 7B shows TYRP1 expression in patients with unpaired extracranial metastases (n=43) or brain metastases (n=29).

FIGS. 8A-8B show quantification of TYRP1-positive (TYRP1+) cells in melanoma brain metastases. FIG. 8A is a bar graph showing the number of TYRP1+ cells per mm² of cancerous tissue in 17 patient samples. FIG. 8B shows examples of tissues with varying levels of TYRP1 expression. NU00722 shows high TYRP1 expression, while NU00276 and NU02899 show medium TYRP1 expression. Arrows point to TYRP1-positive cells. The scale bar is 100 μm.

### DETAILED DESCRIPTION

The present invention provides bispecific antibodies and methods of using the bispecific antibodies to activate a specific cytotoxic immune response against tumor cells.

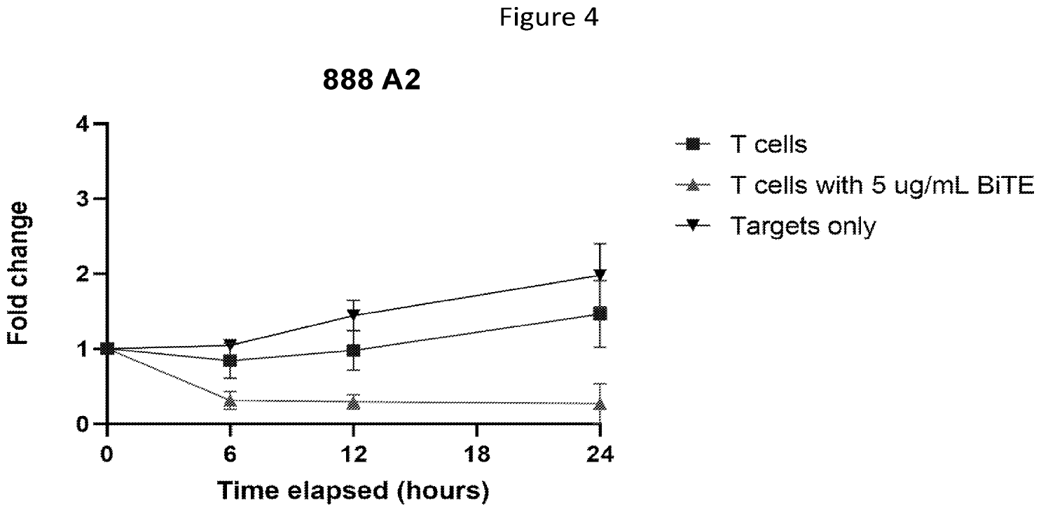

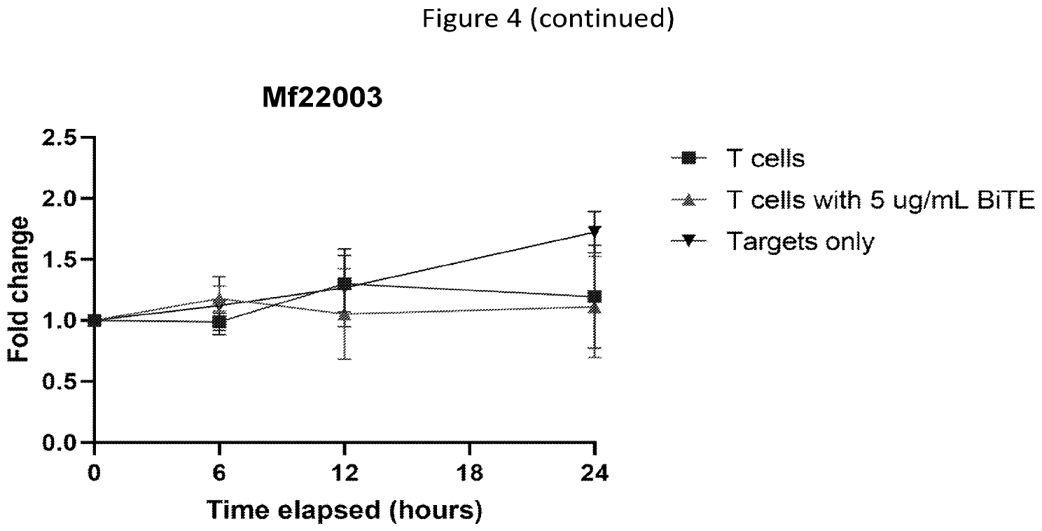

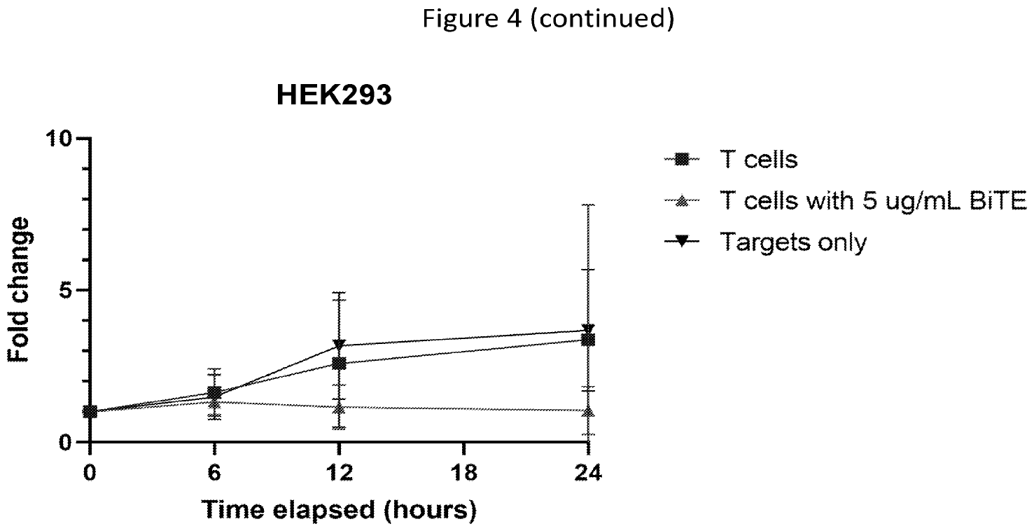

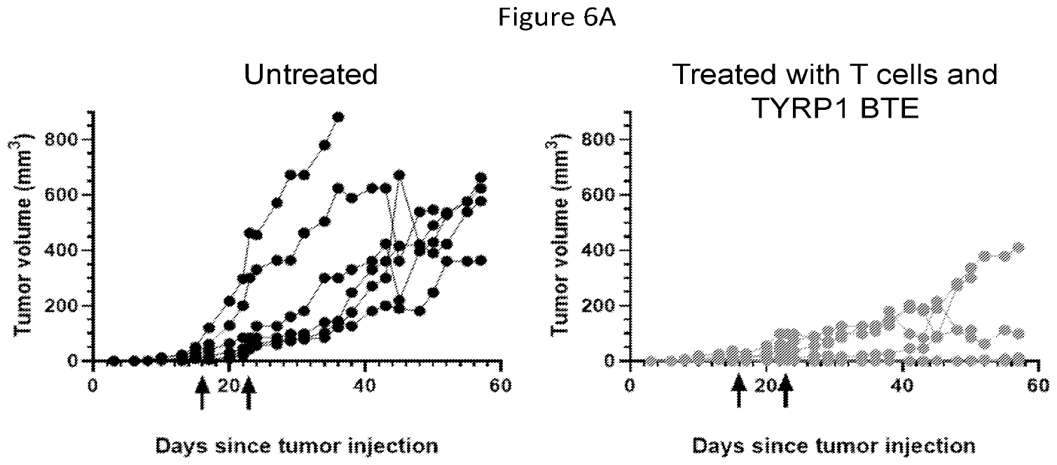

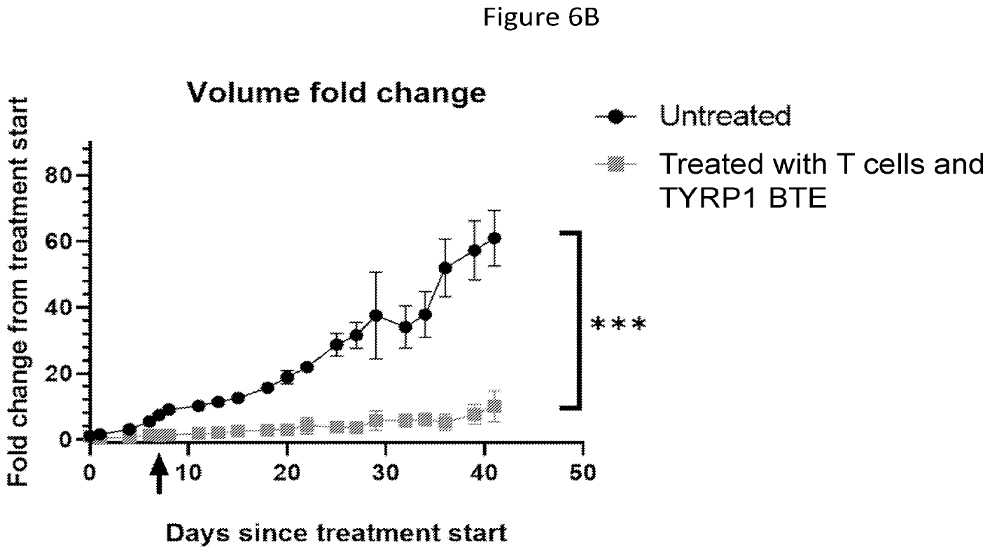

The bispecific antibodies of the present invention are designed to target T cells to melanoma cells. They consist of two single-chain variable fragments (scFvs) linked together. One scFv binds to the T cell co-receptor CD3ε, and the other scFv binds to the protein tyrosinase-related protein 1 (TYRP1), a cell surface marker of melanoma cells. In the Examples, the inventors demonstrate that when their bispecific antibody (which is referred to herein as the TYRP1 BTE) binds to both a T cell and a TYRP1-expressing melanoma cell, it activates the T cell to kill the melanoma cell in a TYRP1-dependent fashion (FIG. 4). Further, they demonstrate that their bispecific antibody can be used to reduce tumor volume in vivo in a mouse model of melanoma (FIG. 6A, FIG. 6B).

One of the primary advantages of using a TYRP1-targeting bispecific antibody to treat melanoma is that these antibodies promote cytotoxicity in a highly targeted manner, causing the destruction of melanoma cells with minimal concern for side effects that result in skin, ocular, auditory, or other abnormalities. The use of bispecific antibodies also overcomes several of the limitations associated with chimeric antigen receptor (CAR) T cells. For example, the use of CAR T cell therapies requires that a patient's T cells are modified and expanded ex vivo prior to use, whereas bispecific antibodies can be supplied as an off-the-shelf reagent that can be used for any patient.

### Bispecific Antibodies:

In a first aspect, the present invention provides bispecific antibodies that target T cells to melanoma cells and promote activation of their cytotoxic activity.

The term “antibody” refers to a protein that comprises at least one antigen-binding domain from an immunoglobulin molecule. As used herein, this term encompasses whole antibodies (e.g., IgG, IgA, IgE, IgM, IgD), chimeric antibodies, and antibody fragments, including single chain variable fragments (scFvs). “Whole” antibodies comprise at least two heavy (H) chains and two light (L) chains. Each heavy chain comprises a heavy chain variable domain (V_{H}) and a heavy chain constant region, and each light chain comprises a light chain variable domain (V_{L}) and a light chain constant region. The variable domains contain a binding domain that interacts with an antigen, while the constant regions may mediate the binding of the antibody to host tissues or factors. The V_{H} and V_{L} each comprise regions of hypervariability, termed complementarity determining regions (CDRs), that are interspersed within regions that are more conserved, termed framework regions (FR). Each V_{H} and V_{L} comprises three CDRs and four FRs, arranged from amino-terminus (N-terminus) to carboxy-terminus (C-terminus) in the following order: FR1, CDR1, FR2, CDR2, FR3, CDR3, FR4.

The term “bispecific” is used to indicate that a molecule can bind specifically to two distinct moieties at the same time. Thus, the term “bispecific antibody” refers to an antibody that is capable of binding to two distinct antigens at the same time. The bispecific antibodies of the present invention are designed to function as bispecific T cell engagers. A “bispecific T cell engager (BTE)” is a bispecific antibody that has one “arm” that binds to a tumor-specific cell surface antigen and a second “arm” that binds to an activating, invariant component of the T cell receptor (TCR) complex, such that simultaneous binding of these moieties by the BTE forces an interaction between a tumor cell and a T cell that results in activation of the T cell and lysis of the tumor cell. Generally, each arm of the BTE is a scFv with the noted specificity. Thus, BTEs are a form of immunotherapy that can be used to treat a tumor if a tumor-specific cell surface antigen can be identified. The terms BTE and bispecific antibody are used interchangeably herein.

The bispecific antibodies described herein comprise or consist of two single-chain variable fragments (scFvs) linked together. As used herein, the term “single-chain variable fragment” (scFv) refers to a fusion protein of the variable domains of the heavy chain (V_{H}) and light chain (V_{L}) of an antibody, connected via a linker peptide. The linker can either connect the N-terminus of the V_{H} with the C-terminus of the V_{L}, or vice versa.

In the Examples, the inventors tested a bispecific antibody that is referred to herein as the “TYRP1 BTE”. This bispecific antibody comprises a first scFv that binds to the activating T cell antigen CD3ε (i.e., the 28F11 scFv) linked to a second scFv that binds to tyrosinase-related protein 1 (TYRP1) (i.e., the TA99 scFv).

CD3 (cluster of differentiation 3) is a T cell co-receptor that is involved in activating both cytotoxic T cells and T helper cells. It is a protein complex that is composed of four distinct chains. In mammals, the complex contains a CD3γ chain, a CD3β chain, and two CD3ε chains. These chains associate with the T-cell receptor (TCR) and the CD3-ζ chain to generate an activation signal in T cells. Thus, by binding to CD3ε, the bispecific antibodies of the present invention activate T cells in a major histocompatibility complex (MHC)-independent manner and are, therefore, unaffected by the MHC downregulation that occurs in some cancers.

Tyrosinase-related protein 1 (TYRP1) is the most abundant protein expressed by melanocytes (i.e., specialized cells that produce the pigment melanin). TYRP1 is also highly expressed in tumors derived from melanocytes, i.e., melanomas. In tumor cells, TYRP1 is trafficked to the cell surface, where it can be recognized by bispecific antibodies. This is evidenced by the fact that the anti-TYRP1 antibody Ta99 binds to intact, non-permeabilized melanoma cells. Thus, cell surface expression of TYRP1 is found preferentially on melanoma cells and can be used as a marker of these cells (Int J Cancer 86(6):818-26, 2000; Pigment Cell Res 8(2):97-104, 1995).

The TYRP1 BTE, i.e., the bispecific antibody that was tested in the Examples, comprises from N-terminus to C-terminus: the V_{H} of the 28F11 scFv (i.e., the CD3ε-binding scFv), a first linker, the V_{L} of the 28F11 scFv, the V_{L} of the TA99 scFv (i.e., the TYRP1-binding scFv), a second linker, and the V_{H} of the TA99 scFv. Thus, in some embodiments, the bispecific antibodies comprise these peptide domains linked in this order. However, the bispecific antibodies may also comprise any other arrangement of these peptide domains.

In some embodiments, the bispecific antibodies comprise complementarity determining regions (CDRs) found in the TYRP1 BTE. A “complementarity determining region (CDR)” is a part of a variable domain of an antibody that binds to a specific antigen. Three CDRs (i.e., CDR1, CDR2, and CDR3) are arranged non-consecutively in each variable domain. Together, the six CDRs of the two variable domains (i.e., V_{H} and V_{L}) form an antigen receptor (i.e., the part of the antibody that binds to the antigen). Thus, in these embodiments, the 28F11 scFv comprises (a) a first V_{H} comprising a CDR1 of SEQ ID NO:5, a CDR2 of SEQ ID NO:6, and a CDR3 of SEQ ID NO:7; (b) a first linker; and (c) a first V_{L} comprising a CDR1 of SEQ ID NO:9, a CDR2 having the amino acid sequence DAS, and a CDR3 of SEQ ID NO:10; and the TA99 scFv comprises (a) a second V_{L} comprising a CDR1 of SEQ ID NO:13, a CDR2 having the amino acid sequence DAK, and a CDR3 of SEQ ID NO:14; (b) a second linker; and (c) a second V_{H} comprising a CDR1 of SEQ ID NO:16, a CDR2 of SEQ ID NO:17, and a CDR3 of SEQ ID NO:18. In some embodiments, the scFvs or portions of them such as the CDRs comprise amino acids that are post-translationally modified. Examples of suitable post-translational modifications include, without limitation, phosphorylation, glycosylation, ubiquitination, nitrosylation, methylation, acetylation, and lipidation.

In some embodiments, the bispecific antibodies comprise whole variable domains found in the TYRP1 BTE. In these embodiments, the first V_{H} comprises SEQ ID NO:4 or an amino acid sequence with at least 90% sequence similarity to SEQ ID NO:4; the first V_{L} comprises SEQ ID NO:8 or an amino acid sequence with at least 90% sequence similarity to SEQ ID NO:8; the second V_{L} comprises SEQ ID NO:12 or an amino acid with at least 90% sequence similarity to SEQ ID NO:12; and/or the second V_{H} comprises SEQ ID NO:15 or an amino acid with at least 90% sequence similarity to SEQ ID NO:15.

“Percentage of sequence similarity’” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Proc. Natl. Acad. Sci. USA (1990) 87: 2267-2268; Nucl. Acids Res. (1997) 25: 3389-3402). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula disclosed in Proc. Natl. Acad. Sci. USA (1990) 87: 2267-2268, which is hereby incorporated by reference in its entirety. The BLAST programs can be used with the default parameters or with modified parameters provided by the user.

In some embodiments, the bispecific antibodies comprise whole scFvs found in the TYRP1 BTE, i.e., scFvs derived from the antibodies 28F11 and TA99. 28F11 is anti-CD3ε antibody fab fragment of human origin. TA99 is a monoclonal antibody of mouse origin that reacts with human and mouse TYRP1. In the TYRP1 BTE, the scFv derived from 28F11 is SEQ ID NO:3 and the scFv derived from TA99 is SEQ ID NO:11. Thus, in these embodiments, the first scFv comprises SEQ ID NO:3 or an amino acid sequence with at least 90% sequence similarity to SEQ ID NO:3; and/or the second scFv comprises SEQ ID NO:11 or an amino acid sequence with at least 90% sequence similarity to SEQ ID NO:11.

In some embodiments, the bispecific antibodies comprise the entire sequence of the TYRP1 BTE, i.e., SEQ ID NO:2. The sequences of the various portions of this bispecific antibody are outlined in Table 1, below.

In the TYRP1 BTE tested in the Examples, the 28F11 scFv comprises a V_{H} CDR2 of SEQ ID NO:23 (IWYDGSKK) and the Ta99 comprises a V_{H} CDR2 of SEQ ID NO:25 (INPDNGNT). However, the inventors have identified G to A amino acid substitution mutations that may render these CDRs more resistant to degradation. Thus, in some embodiments, the 28F11 scFv comprises a V_{H} CDR2 of SEQ ID NO:24 (IWYDASKK) and the Ta99 comprises a V_{H} CDR2 of SEQ ID NO:26 (INPDNANT). Note: SEQ ID NO:6 (IWYDXSKK) encompasses both versions of the 28F11 scFv V_{H} CDR2 sequence (i.e., both SEQ ID NO:23 and SEQ ID NO:24), and SEQ ID NO:17 (INPDNXNT) encompasses both versions of the Ta99 scFv V_{H} CDR2 sequence (i.e., both SEQ ID NO:25 and SEQ ID NO:26).

TABLE 1
TYRP1 BTE sequences

| | | variable | |
|---|---|---|---|
| Whole BTE | scFvs | domains | CDRs |
| SEQ ID | 28F11 scFv | V_{H} | CDR1 |
| NO: 2 | SEQ ID NO: 3 | SEQ ID NO: 4 | SEQ ID NO: 5 |
| | | | CDR2 |
| | | | SEQ ID NO: 6 |
| | | | (=SEQ ID NO: 23 |
| | | | or SEQ ID NO: 24) |
| | | | CDR3 |
| | | | SEQ ID NO: 7 |
| | | V_{L} | CDR1 |
| | | SEQ ID NO: 8 | SEQ ID NO: 9 |
| | | | CDR2 |
| | | | DAS |
| | | | CDR3 |
| | | | SEQ ID NO: 10 |
| | TA99 scFv | V_{L} | CDR1 |
| | SEQ ID NO: 11 | SEQ ID NO: 12 | SEQ ID NO: 13 |
| | | | CDR2 |
| | | | DAK |
| | | | CDR3 |
| | | | SEQ ID NO: 14 |
| | | V_{H} | CDR1 |
| | | SEQ ID NO: 15 | SEQ ID NO: 16 |
| | | | CDR2 |
| | | | SEQ ID NO: 17 |
| | | | (=SEQ ID NO: 25 |
| | | | or SEQ ID NO: 26) |
| | | | CDR3 |
| | | | SEQ ID NO: 18 |

As used herein, the term “linker peptide” refers to a peptide that bridges together two polypeptide segments within a fusion protein. A linker peptide may comprise 1, 2, 3, 4, 5, 6, 7, 8, 9, 10, 11, 12, 13, 14, 15, 16, 17, 18, 19, 20, or more amino acid residues. The linkers used in the bispecific antibodies of the present invention may comprise any amino acid sequence that does not substantially hinder the interaction of the two scFvs with their corresponding target molecules. In some embodiments, the linker is flexible such that it has no required fixed structure in solution and the adjacent polypeptides are free to move relative to one another. Preferred amino acid residues for flexible linker sequences include glycine, alanine, serine, threonine, lysine, arginine, glutamine, and glutamic acid. In some embodiments, the linker peptide is a glycine-serine linker (i.e., a linker comprising the amino acids glycine and serine).

In the bispecific antibodies of the present invention, the V_{H} and V_{L} domains of the first scFv are linked together by a first linker, and the V_{H} and V_{L} domains of the second scFv are linked together by a second linker. In preferred embodiments, the first linker and second linker are 10-20 amino acid glycine-serine linkers. The inventors used the 15-amino-acid (Gly4S)₃ linker of SEQ ID NO:19 as both the first linker and second linker in the TYRP1 BTE. Thus, in some embodiments, the first linker and/or second linker is the (Gly4S)₃ linker of SEQ ID NO:19.

In some embodiments, the first scFv and the second scFv are linked via a third linker. In preferred embodiments, the third linker is a 20-30 amino acid glycine-serine linker. The inventors used the 23-amino acid glycine-serine linker of SEQ ID NO:20 to link the first and second scFv together in the TYRP1 BTE. Thus, in some embodiments, the third linker is the linker of SEQ ID NO:20.

The inventors included the signal peptide of SEQ ID NO:21 on the 5′ end of the TYRP1 BTE. Thus, in some embodiments, the bispecific antibodies further comprise a signal peptide on the 5′ end. A “signal peptide” is a peptide that allows for cellular secretion of an antibody into the extracellular space. Signal peptides are cleaved off following secretion during maturation of the antibody. In preferred embodiments, the signal peptide is SEQ ID NO:21. However, many other suitable signal peptides are known in the art.

Additionally, the inventors included a tag on the 3′ end of the TYRP1 BTE. Thus, in some embodiments, the bispecific antibodies further comprise a tag. As used herein, a “tag” is a peptide that is genetically grafted onto a fusion protein for a particular purpose. Suitable tags for use in the bispecific antibodies of the present invention include, without limitation, affinity tags for protein purification (e.g., chitin binding protein (CBP), maltose binding protein (MBP), Strep, glutathione-S-transferase (GST), poly(His) tags), solubilization tags (e.g., thioredoxin (TRX), poly(NANP), MBP, GST), epitope tags for antibody-based detection (e.g., ALFA-tag, V5-tag, Myc-tag, HA-tag, Spot-tag, T7-tag, NE-tag), and fluorescent tags (e.g., green fluorescent protein (GFP), red fluorescent protein (RFP)), and enzymatic tags (e.g., horseradish peroxidase, alkaline phosphatase, beta-galactosidase, glucose-6-phosphatase, acetylcholinesterase) for visual detection. The inventors used the 6×His tag of SEQ ID NO:12 in the TYRP1 BTE. Thus, in some embodiments, the tag is the 6×His tag.

### Pharmaceutical Compositions:

In a second aspect, the present invention provides pharmaceutical compositions comprising a bispecific antibody described herein and a pharmaceutically acceptable carrier.

The term “pharmaceutically acceptable carrier” refers to any carrier, diluent, or excipient that is compatible with the other ingredients of a formulation and is not deleterious to a recipient to which it is administered. Pharmaceutically acceptable carriers are known in the art and include, but are not limited to, diluents (e.g., Tris-HCl, acetate, phosphate), preservatives (e.g., thimerosal, benzyl alcohol, parabens), solubilizing agents (e.g., glycerol, polyethylene glycerol), emulsifiers, liposomes, nanoparticles, and adjuvants. Pharmaceutically acceptable carriers may be aqueous or non-aqueous solutions, suspensions, or emulsions. Examples of nonaqueous solvents are propylene glycol, polyethylene glycol, vegetable oils (e.g., olive oil), and injectable organic esters (e.g., ethyl oleate). Aqueous carriers include isotonic solutions, alcoholic/aqueous solutions, emulsions, and suspensions, including saline and buffered media.

The compositions of the present invention may further include additives such as albumin or gelatin to prevent absorption to surfaces, detergents (e.g., Tween 20, Tween 80, Pluronic F68, bile acid salts), antioxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

### Nucleic Acids:

In a third aspect, the present invention provides nucleic acids encoding a bispecific antibody described herein.

The terms “nucleic acid,” “polynucleotide,” and “oligonucleotide” are used interchangeably to refer to a polymer of DNA or RNA. A nucleic acid may be single-stranded or double-stranded and may represent the sense or the antisense strand. A nucleic acid may be synthesized or obtained from a natural source. A nucleic acid may contain natural, non-natural, or altered nucleotides, as well as natural, non-natural, or altered internucleotide linkages (e.g., phosphoroamidate linkages, phosphorothioate linkages).

In some embodiments, the nucleic acids comprise SEQ ID NO:1, i.e., the codon-optimized sequence used to encode the TYRP1 BTE for expression and purification in the Examples.

### Vectors:

In a fourth aspect, the present invention provides vectors comprising a nucleic acid described herein.

The term “vector” refers to a DNA molecule that is used to carry a particular DNA segment (i.e., a DNA segment included in the vector) into a host cell. Some vectors are capable of autonomous replication in a host cell into which they are introduced (e.g., bacterial vectors that include a bacterial origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product. Vectors may also include a reporter gene or a selectable marker gene. Suitable vectors include plasmids (i.e., circular double-stranded DNA molecules) and mini-chromosomes.

In some embodiments, the vector is an expression vector. An “expression vector” is a vector that comprises a sequence encoding a gene product (e.g., a bispecific antibody described herein) operatively linked to a regulatory element (e.g., a promoter) that drives expression of the gene product in a cell. The expression vector may contain one or more transcriptional unit (i.e., coding sequence/regulatory element combination). The expression vector may also include additional sequences (e.g., a sequence encoding a signal peptide or a tag) that modify the encoded gene product.

In the Examples, the inventors cloned cDNA encoding the TYRP1 BTE into a lentiviral vector and used it to transfect HEK293T/17 cells for expression and purification of the TYRP1 BTE. Thus, in some embodiments, the vector is a lentiviral vector.

### Transgenic Cells:

In a fifth aspect, the present invention provides transgenic cells that express a bispecific antibody described herein.

A “transgenic cell” is a cell that contains genetic material into which DNA from an unrelated organism has been artificially introduced. The transgenic cells of the present invention may comprise a vector described herein that encodes the bispecific antibody.

### Methods of Treating Tumors:

In a sixth aspect, the present invention provides methods of treating a TYRP1-expressing tumor in a subject. The methods comprise administering a therapeutically effective amount of a bispecific antibody, pharmaceutical composition, or transgenic cell described herein to the subject to treat the tumor.

As used herein the term “tumor” refers to an abnormal mass of tissue in which the growth of the mass surpasses and is not coordinated with the growth of normal tissue. This term also encompasses blood or other bodily fluid containing cancerous cells. A tumor can be characterized as “benign” or “malignant” depending on the following characteristics: degree of cellular differentiation, rate of growth, local invasion, and metastasis. A “benign” tumor is often well differentiated, has characteristically slower growth than a malignant tumor, and remains localized to the site of its origin. In some cases, a benign tumor does not have the capacity to infiltrate, invade, or metastasize to distant sites. A “malignant” tumor is often poorly differentiated and has characteristically rapid growth accompanied by progressive infiltration, invasion, and destruction of the surrounding tissue. Furthermore, a malignant tumor can have the capacity to metastasize to distant sites.

The bispecific antibodies of the present invention are designed to target TYRP1. TYRP1 expression is enriched on the surface of melanoma cells as compared to on the surface of healthy human cells, such as melanocytes. (Note: TYRP1 is expressed at very low levels on the surface of melanocytes. In these cells, TYRP1 expression is primarily intracellular.) TYRP1 expression has also been detected in glioma (Am J Pathol 150(6):2143-52, 1997) and in lymphangioleiomyomatosis (Am J Pathol 175(6):2463-72, 2009), but this protein is thought to be expressed at very low levels in other cancers.) Thus, in preferred embodiments, the tumor is a melanoma. “Melanoma” is a type of cancer that develops from the pigment-producing cells known as melanocytes. Melanomas typically occur in the skin, but may rarely occur in the mouth, intestines, or eye. As used herein, this term includes both cutaneous and metastatic melanoma. Because melanoma tumors are commonly infiltrated by T cells, they are a suitable target for bispecific antibody-based immunotherapy.

In some embodiments, the methods further comprise determining whether TYRP1 is expressed on a tumor cell in the subject. In these embodiments, the bispecific antibody, pharmaceutical composition, or transgenic cell is only administered to the subject if TYRP1 expression is detected. To assay for TYRP1 expression, a tissue sample or biopsy may be collected and analyzed using any standard method for detecting gene or protein expression. Suitable methods include, without limitation, Northern blot, western blot, in situ hybridization, immunohistochemistry, immunocytochemistry, reverse transcription polymerase chain reaction, microarray, RNA sequencing, and the like.

As used herein, “treating” describes something that is done to a subject to combat a health problem (i.e., a disease, condition, or disorder). Treating may involve controlling the health problem, lessening its symptoms or complications, and/or eliminating it. For example, treating a tumor in a subject includes reducing the volume of a tumor, reducing the number of tumor cells within the subject, and reducing, repressing, delaying, or preventing the growth or metastasis of the tumor. Accordingly, as used herein, the term “therapeutically effective amount” refers to an amount sufficient to achieve one or more of these outcomes. For any active agent, a therapeutically effective amount can be estimated initially in cell culture assays or in an animal model.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent.

Methods for determining an effective means of administration and dosage are well known to those of skill in the art and will vary with the formulation used for the treatment, the purpose of the treatment, the target cell being treated, and the subject being treated. Single or multiple administrations can be carried out with the dose level and pattern selected by the treating physician.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

### Methods of Inducing Lysis of a Target Cell:

In a seventh aspect, the present invention provides methods for inducing lysis of a TYRP1-expressing target cell. The methods comprise contacting the target cell with a bispecific antibody described herein in the presence of a T cell in an amount effective to lyse the target cell.

A “T cell” is a type of lymphocyte that is characterized by the presence of a T-cell receptor (TCR) on its cell surface. T cells play a central role in the adaptive immune response. “Cytotoxic T cells”, which are also known as “killer T cells” or “CD8+ T cells”, can directly kill target cells bearing specific antigens (e.g., tumor-associated antigens) while sparing neighboring healthy cells. Thus, in preferred embodiments, the T cell is a cytotoxic T cell.

The term “lysis” refers to a process in which the membrane of a cell is broken down. Cytotoxic T cells can lyse target cells via two independent pathways: (1) exocytosis of granules containing perforin, and (2) interaction of Fas ligand on the T cell with Fas on the target cell.

The target cell that is lysed by these methods may be any cell that expresses TYRP1 on its surface. However, in preferred embodiments, the target cell is a tumor cell, and the target cell is contacted with the bispecific antibody in vivo in a subject that has a tumor.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

### Example 1

In the following example, the inventors describe experiments that were used to validate the tyrosinase-related protein 1 (TYRP1) bispecific T-cell engager (BTE) described herein.

### Materials and Methods

### Design and Generation of the TYRP1 BTE Construct

The TYRP1 BTE consists of two scFvs linked by 23-amino acid flexible linker. One scFv, an scFv derived from the antibody 28F11 (i.e., Foralumab (28F11-AE; NI-0401), developed by Novimmune), targets CD3ε on T cells. The other scFv, an scFv derived from the antibody TA99, targets TYRP1 on tumor cells. The heavy and light chains of TA99 used in the TA99 scFv are those described by Boross et al. (Immunol Lett 160(2):151-7, 2014), except that an aspartic acid (D) was added to the N-terminus of the light chain. Within the TYRP1 BTE, 28F11 scFv is arranged in the V_{H}-V_{L} orientation (i.e., N-terminus to C-terminus) and TA99 scFv is ranged in the V_{L}-V_{H} orientation (i.e., N-terminus to C-terminus). The V_{H} and V_{L} domains of each scFv are connected by a (Gly4S)₃ flexible linker. The 28F11 scFv is on the N-terminal end of the BTE, and the TA99 scFv is on the C-terminal end of the BTE.

The cDNA encoding the TYRP1 BTE was codon optimized for expression in human cells. GenScript synthesized the codon-optimized cDNA and subcloned it into the pLVX-IRES-zsGreen1 lentiviral vector (Takara Bio, cat #632187). The whole plasmid has been sequenced to validate the final product.

Generation of a Cell Line that Produces the TYRP1 BTE

To generate cells that produce secretable TYRP1 BTE, lentiviral particles were first generated. HEK293T/17 cells (ATCC, cat #CRL-11268) were transfected with the pLVX-IRES-zsGreen1 lentiviral vector encoding the TYRP1 BTE using the Lenti-X™ Packaging Single Shots transfection/packaging system according to manufacturer protocol (Takarabio, Cat. No. 631276). After 48 hours, supernatant containing lentiviral particles was collected, cleared by centrifugation at 40 g for 5 minutes, and used for transduction of HEK293T/17 cells in the presence of 8 μg/ml polybrene. To enrich the population of transduced cells, cells were sorted for zsGreen1 signal and were then expanded and cryopreserved.

### Generation of Recombinant TYRP1 BTE Protein

The transduced HEK293T/17 cells were expanded in Dulbecco's Modified Eagle Medium (DMEM) media supplemented with 10% fetal bovine serum (FBS) and penicillin-streptomycin at 37° C. in 5% CO₂ to generate the recombinant protein for downstream analysis. Flasks containing the transduced HEK293T/17 cells were placed in an incubator with 5% CO₂ and cultured at 32° C. for 48-72 hours. The supernatants were then collected, centrifuged at 400 g for 5 minutes, and filtered through 0.45 μm filters. The supernatants were then incubated overnight with cOmplete His-Tag purification resin (Roche Diagnostics GmbH, Cat. No. 52000300) on a rotating shaker at 4° C. to capture the TYRP1 BTE via its 6×His tag. The next day, the TYRP1 BTE was purified according to the manufacturer's protocol. TYRP1 BTE protein was eluted from the column and was dialyzed against phosphate-buffered saline pH 7.4, aliquoted, and stored at −80° C.

### Western Blot

Protein samples were prepared in Laemmli Sample Buffer (Bio-Rad, Hercules, CA; Cat. No. 1610747) with 2.5% 2-mercaptoethanol (Sigma Aldrich, St. Louis, MO; Cat. No. M6250). The denatured protein samples were then incubated at 95° C. for 5 minutes, whereas the native protein samples were not. Protein samples (500 ng per lane) and 250 kDa Precision Plus Protein standard (Bio-Rad Laboratories, Hercules, CA) were loaded into wells of a 4-20% Midi-PROTEAN® TGX Stain-Free™ gel (Bio-Rad). Proteins were separated by size using sodium dodecyl-sulfate polyacrylamide gel electrophoresis (SDS-PAGE) and transferred onto a methanol-activated 0.2 μm polyvinylidene difluoride (PVDF) membrane (Millipore Sigma, Burlington, MA; Cat. No. IPVH08100) using the Trans-Blot® Turbo Transfer System™ (Bio-Rad). Membranes were blocked with 5% non-fat dry milk (NFDM) in tris-buffered saline plus 0.1% Tween-20 (TBST) (Boston Bioproducts, Ashland, MA; Cat. No. IBB180) for 2 hours at room temperature. Membranes were incubated with anti-6×His tag polyclonal antibody (1:5000) (Abcam, Cat. No. Ab1187) in 5% NFDM-TBST overnight at 4° C. The next day, membranes were washed with TBST and incubated with Clarity™ Western ECL Substrate (Bio-Rad). Antibody binding was detected via chemiluminescent imaging using the ChemiDoc™ MP Imaging System (Bio-Rad).

### Cell Culture

HEK293 cells were obtained as CRL-1573 from the American Type Tissue Collection and were maintained in high-glucose DMEM media (Corning) supplemented with 10% FBS (R&D Systems), 1% Antibiotic-Antimycotic (Gibco), and 2 mM L-glutamine (Gibco).

Mf22003 melanocytes were generated from fresh, otherwise discarded skin tissue and were maintained in Medium 254 (Gibco) supplemented with Human Melanocyte Growth Supplement-2 (Gibco), 1% Antibiotic-Antimycotic (Gibco), and 2 mM L-glutamine (Gibco).

888 A2 melanoma cells were acquired from the ATCC and were maintained in RPMI 1640 media (Corning) supplemented with 10% FBS (R&D Systems), 1% Antibiotic-Antimycotic (Gibco), 2 mM L-glutamine (Gibco), 1% MEM Nonessential Amino Acids (Corning), 1 mM Sodium Pyruvate (Corning), 10 mM HEPES (Corning), and 100 μM 2-Mercaptoethanol (Sigma).

Human T cells were cultured in RPMI media (Corning) supplemented with 10% FBS (R&D Systems), 2 mM L-glutamine (Gibco), lx antibiotic-antimycotic (Gibco), 1× Non-Essential Amino Acids (Corning), 1 mM sodium pyruvate (Gibco), 10 mM HEPES (Gibco), 50 μM β-mercaptoethanol (Sigma-Aldrich), and 100 iu/mL rhIL-2 (Hoffmann-La Roche Inc). Human T cells were also expanded using ImmunoCult™-XF T Cell Expansion Medium (Stemcell Technologies) supplemented with 1% Antibiotic-Antimycotic (Gibco), 100 iu/mL of rhIL-2 (Hoffmann-La Roche Inc), Human T-Activator cocktail, and CD3/CD28 Dynabeads (Gibco).

### T Cell Co-Culture

T cells were co-cultured with 888 A2 melanoma cells, Mf22003 melanocytes, or HEK 293 control cells at a 10:1 effector:target ratio. Recombinant TYRP1 BTE protein was added to the coculture at various concentrations (1.25 μg/mL, 2.5 μg/mL, and 5 μg/mL). Supernatant from the co-cultures was saved and stored at −20° C. until ready for use.

### Fluorescence-Activated Cell Sorting (FACS)

Cultured melanocytes (Mf0978) and melanoma cells (B16.F10 and 624.38) were detached using 5 mM EDTA (Invitrogen) in Dulbecco's Phosphate Buffered Saline (PBS) without calcium or magnesium (Corning). Cells were washed and incubated with or without anti-human TYRP1 antibody (Ta99, Biolegend) at a 1:50 dilution in PBS with 2% FBS with Live/Dead Near-IR marker (Life Technologies) at a 1:1000 dilution for 30 minutes at room temperature. Cells were washed and incubated in goat anti-mouse IgG, IgM, IgA FITC (Southern Biotech) at 10 μg/mL in PBS with 2% FBS for 30 minutes at room temperature. Cells were then washed and fixed in Fix/Perm buffer (Biolegend) for 20 minutes at 4° C., followed by another wash, suspension in PBS with 2% FBS, and storage in 4° C. Cells were analyzed via FACS on a Fortessa Cell Analyzer (Becton Dickinson), and gating was performed using FlowJo.

888 A2 melanoma cells were detached using 10 mM EDTA in PBS and stained using anti-human TYRP1 primary antibody (Ta99, Biolegend) followed by Alexa Fluor 488-labeled goat anti-mouse IgG2a secondary antibody (Invitrogen). Cytometry was performed on the Cytek Aurora Spectral Flow Cytometer (Cytek, Fremont, CA) and analyzed using FlowJo version 10 software (BD Biosciences, San Jose, CA).

### IFN-γ Enzyme-Linked Immunosorbent Assay (ELISA)

IFN-γ concentration was measured using a Human IFN-γ ELISA Flex kit (Mabtech). An ELISA plate (R&D Systems) was coated overnight at 4° C. with mAB 1-D1K capture antibody at 2 μg/mL in PBS. Blocking was performed for 1 hour at room temperature using incubation buffer made with PBS, 0.05% Tween-20, and 0.1% bovine serum albumin (BSA). Standards and samples (diluted 1:10 in incubation buffer) were incubated on the plate for 2 hours at room temperature. Detection antibody mAB 7-B6-1-biotin was diluted to 1 μg/mL in incubation buffer and was incubated for 1 hour at room temperature. Streptavidin-HRP was diluted 1:1000 in incubation buffer and was incubated for 1 hour at room temperature. TMB Substrate Solution (Invitrogen) was added to develop the wells for 10 minutes, and Stop Solution (Thermo Scientific) was used to halt the reaction. Absorption was read at 490 nm with a 605 nm correction on a Cytation 3 Imaging Reader (Biotek). A standard curve was generated using GraphPad Prism software and was used to interpolate sample concentrations.

### Incucyte Analysis

Target cells were detached using TrypLE Express Enzyme (Gibco) and stained using 0.30 μM Cytolight Rapid Green Dye (Sartorius). Cells were seeded in TC-treated 96-well microplates (Corning) at 10,000 cells per well in 100 μL in triplicate for each experimental condition. One day after seeding, cell media was replaced with RPMI-based T cell media containing T cells at a density of 100,000 T cells per well, with or without TYRP1 BTE protein diluted 5 μg/mL to a total volume of 100 μL. Plates were placed in an Incucyte S3 imager (Sartorius) and phase-contrast and fluorescent images of each well were taken every 3 hours for up to 48 hours. Live cells were counted manually at each time point.

### Multiplex Cytokine Analysis

Co-culture supernatant was tested using a V-Plex Human Proinflammatory Panel 1 kit (Meso Scale Diagnostics, Rockville, MD) and read on a MESO QuickPlex SQ 120MM machine (Meso Scale Diagnostics). Prism Software (Graphpad) was used to generate standard curves for each analyte and interpolate concentrations of each analyte for each sample.

### Mouse Model Analysis

888 A2 melanoma cells were suspended in HBSS (Gibco) with 8.3% Growth Factor-Reduced Matrigel (Corning) at 30 million cells per milliliter. 3 million cells were injected subcutaneously into the right flanks of SCID-Bg immunodeficient mice. Tumor size was checked 3 times per week until the endpoint. 16 days after tumor cell injection, mice were given retro-orbital injections of 3 million T cells with 75 μg of TYRP1 BTE or were left untreated. Mice were given repeat treatments 1 week after the initial treatment. Following initial treatment, T cell treated mice were given intra-peritoneal injections of 30,000 IU of rhIL-2 3 times per week. Mice were euthanized 43 days after the first treatment date.

### Results

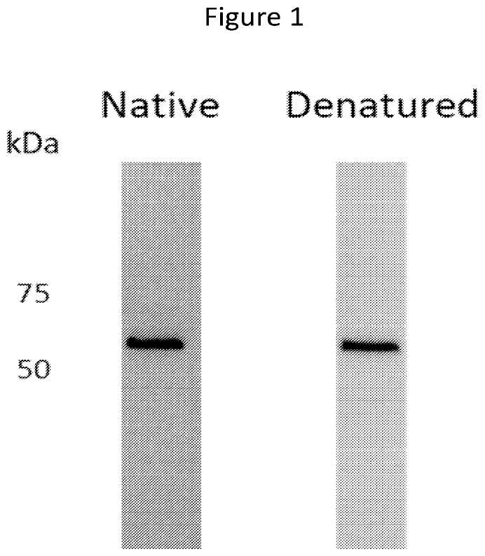

To validate the successful expression and purification of the TYRP1 BTE, a western blot analysis was performed (FIG. 1). In this experiment, the BTE was detected using an antibody that specifically binds to the 6×His tag that was included on the C-terminus of the BTE.

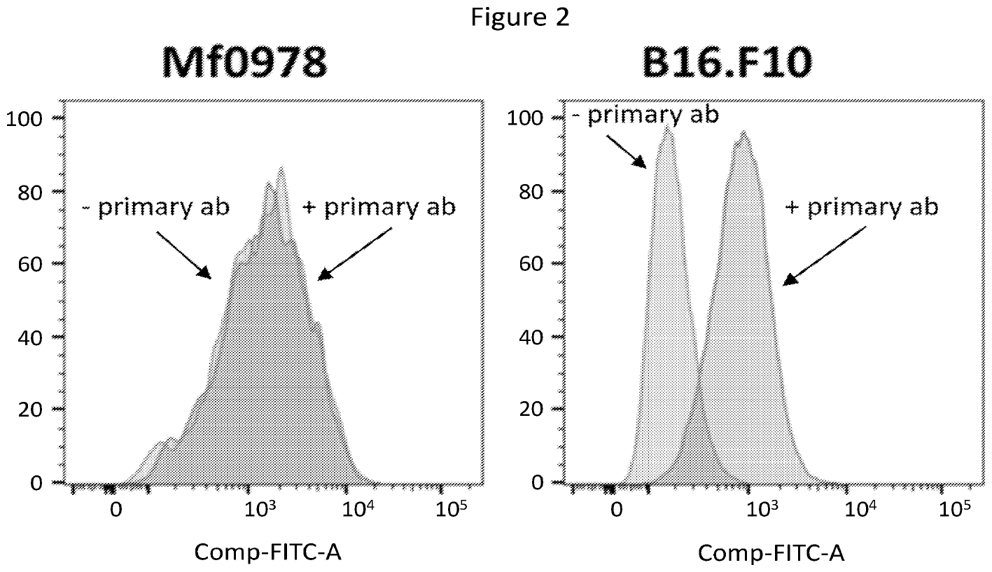

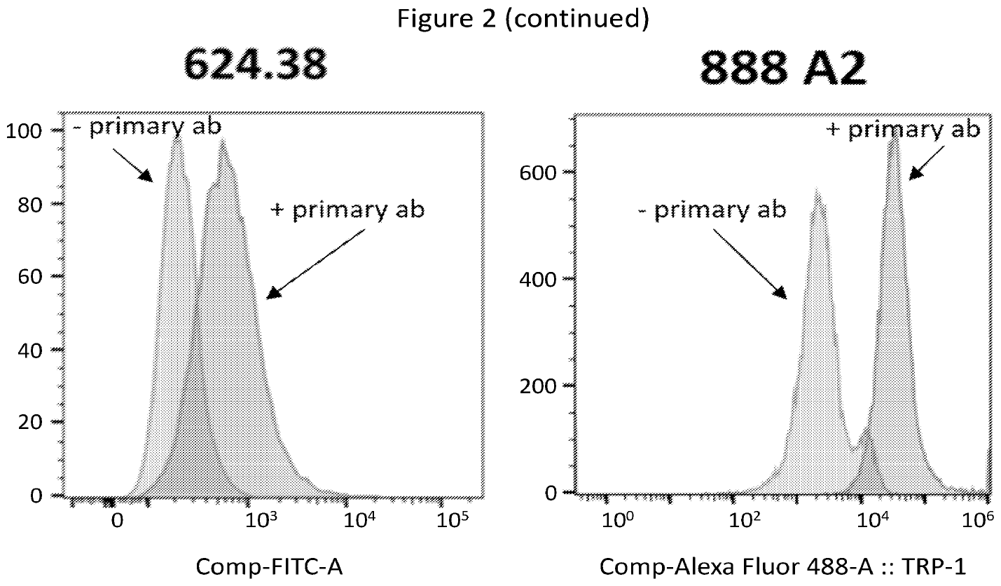

TYRP1 surface expression was confirmed in mouse melanoma cells (B16.F10) and human melanoma (624.38 and 888 A2) via a FACS analysis, in which Mf0978 cultured human early passage melanocytes served as a negative control. The cultured melanoma cells showed markedly greater surface expression of TYRP1 than the primary melanocytes (FIG. 2).

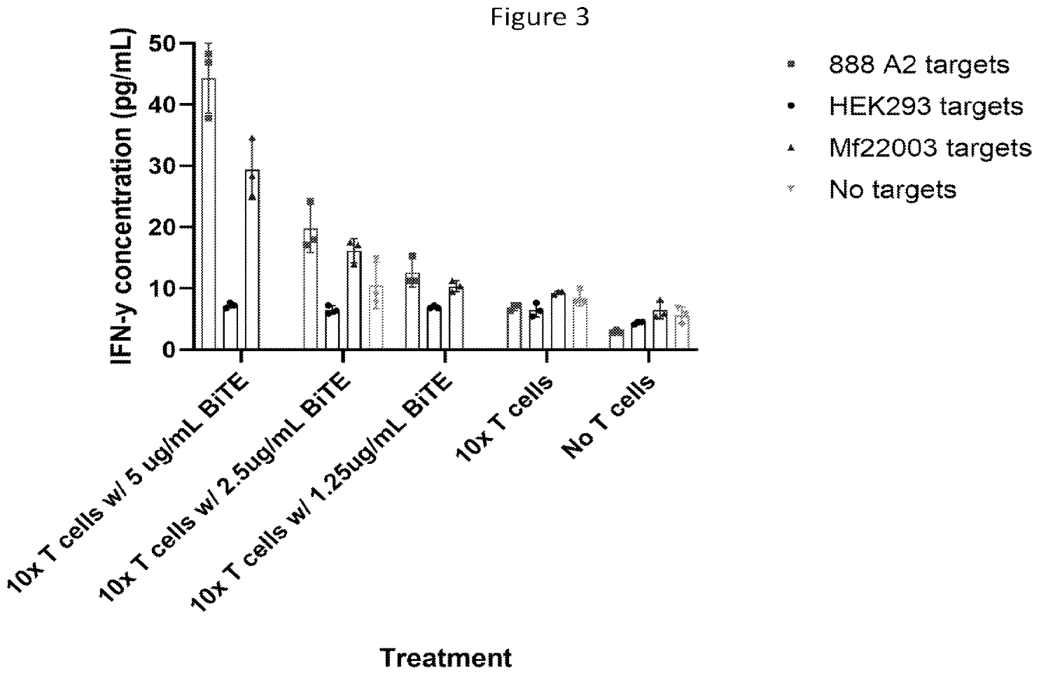

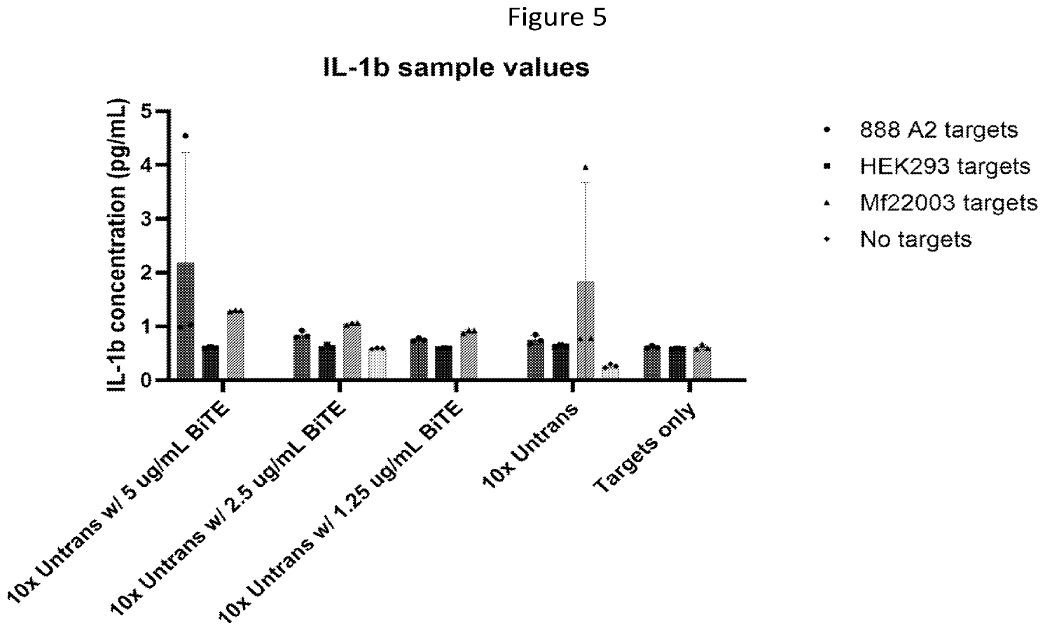

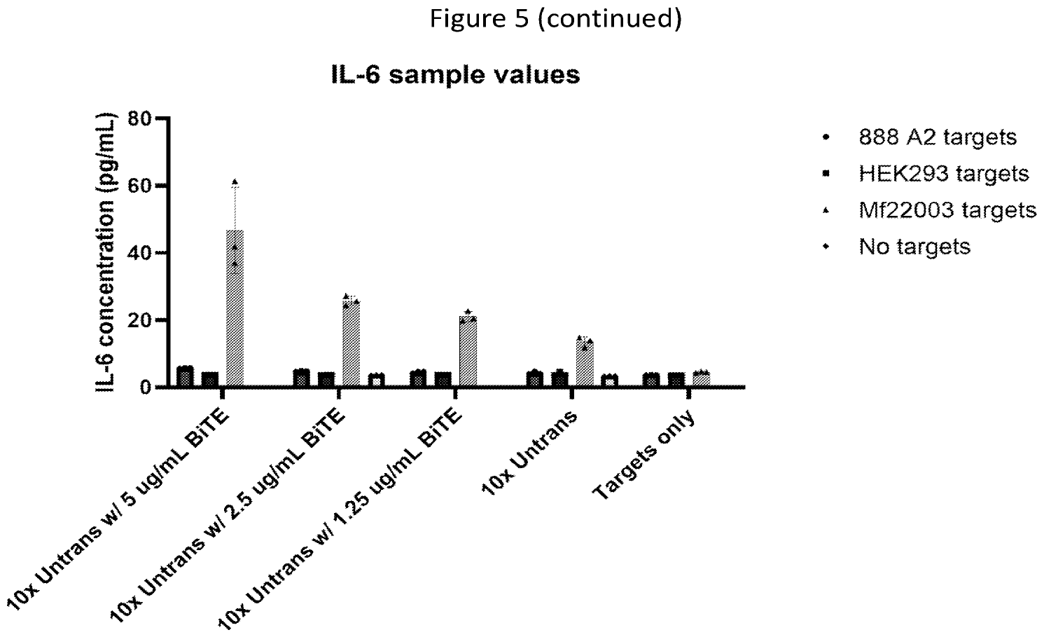

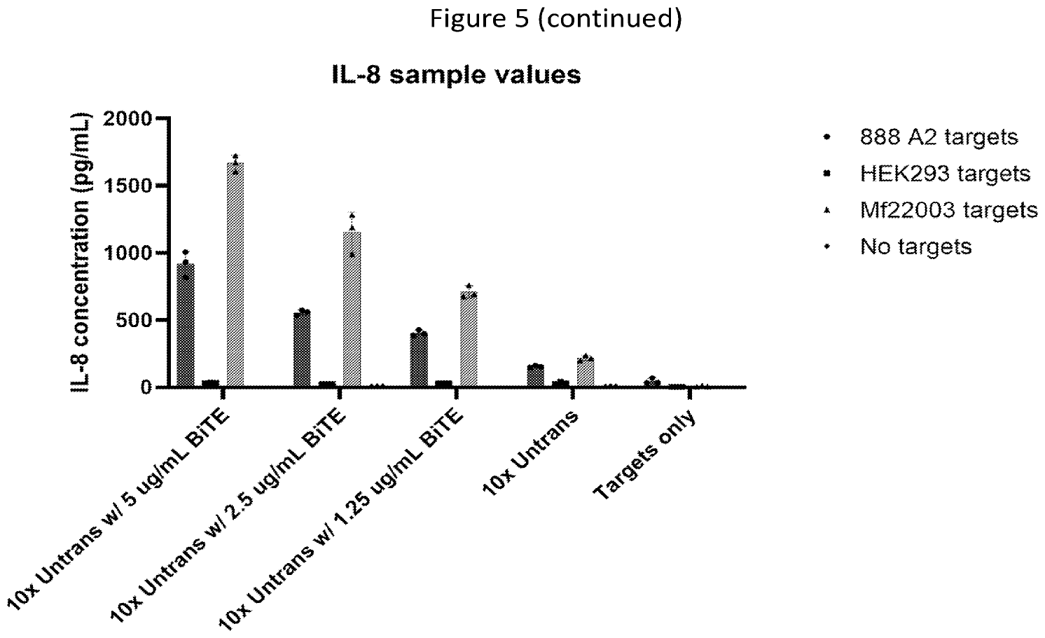

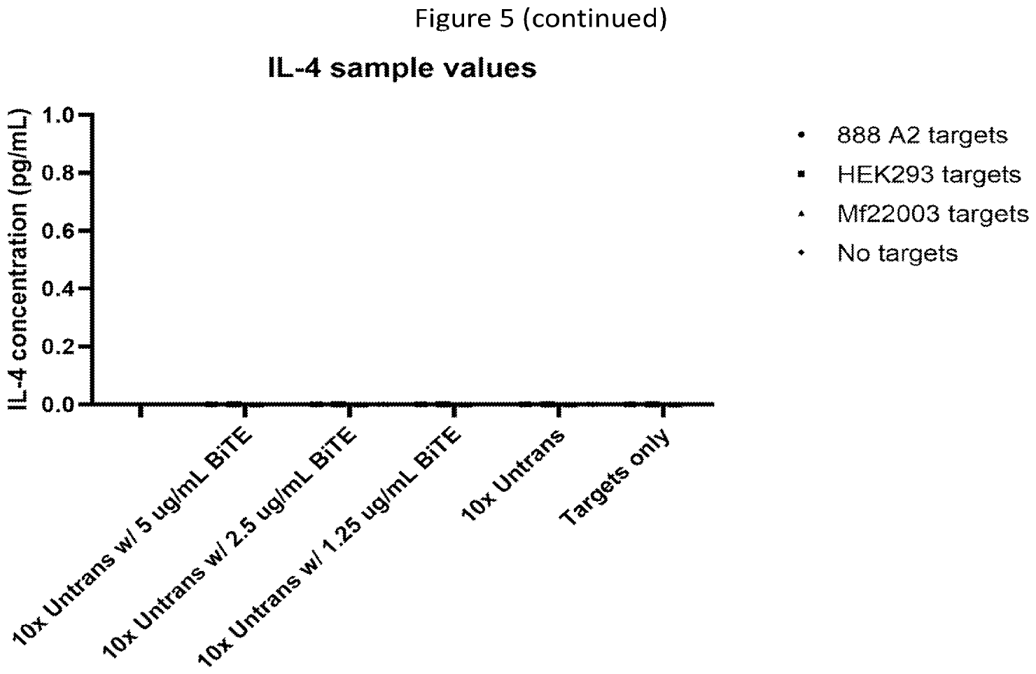

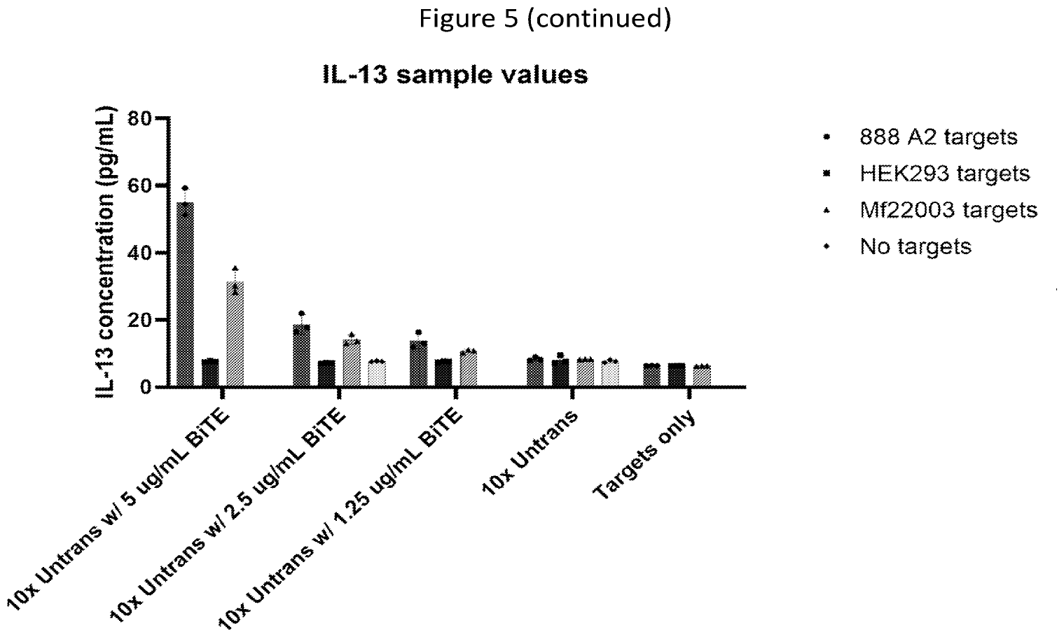

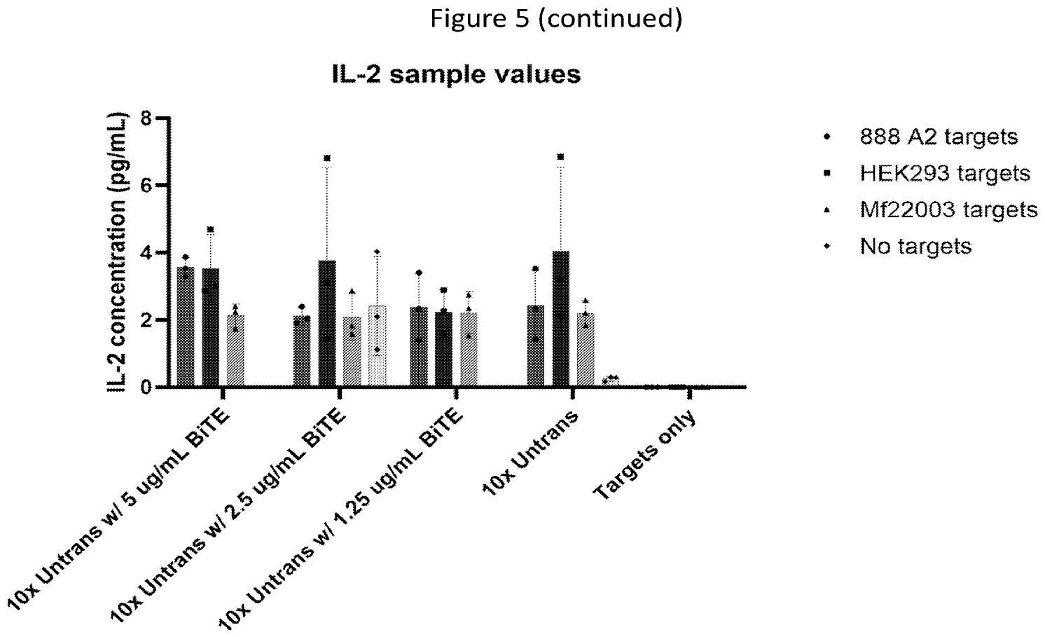

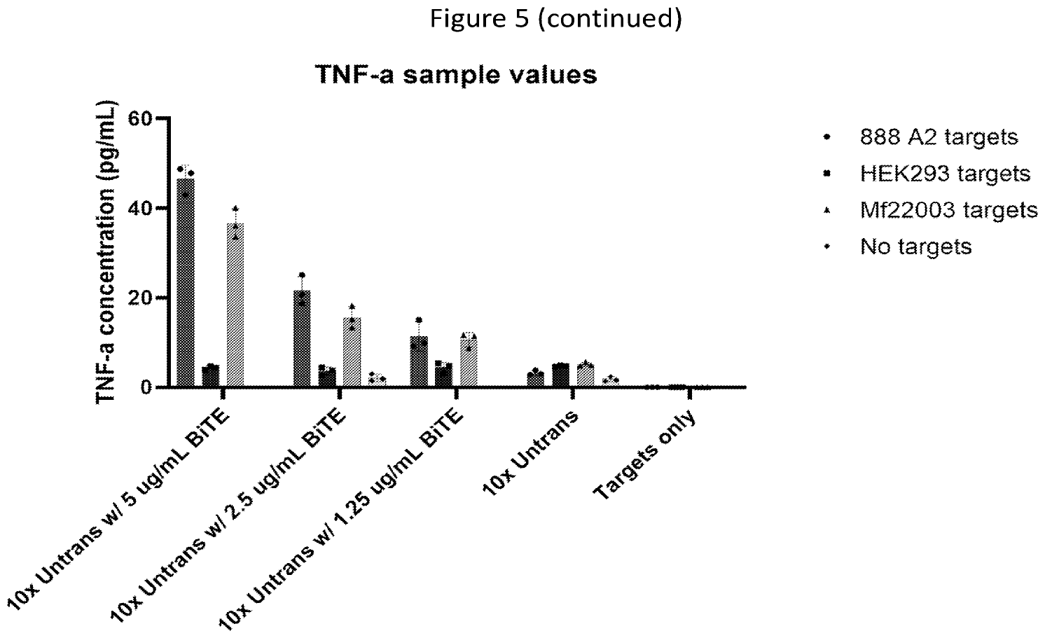

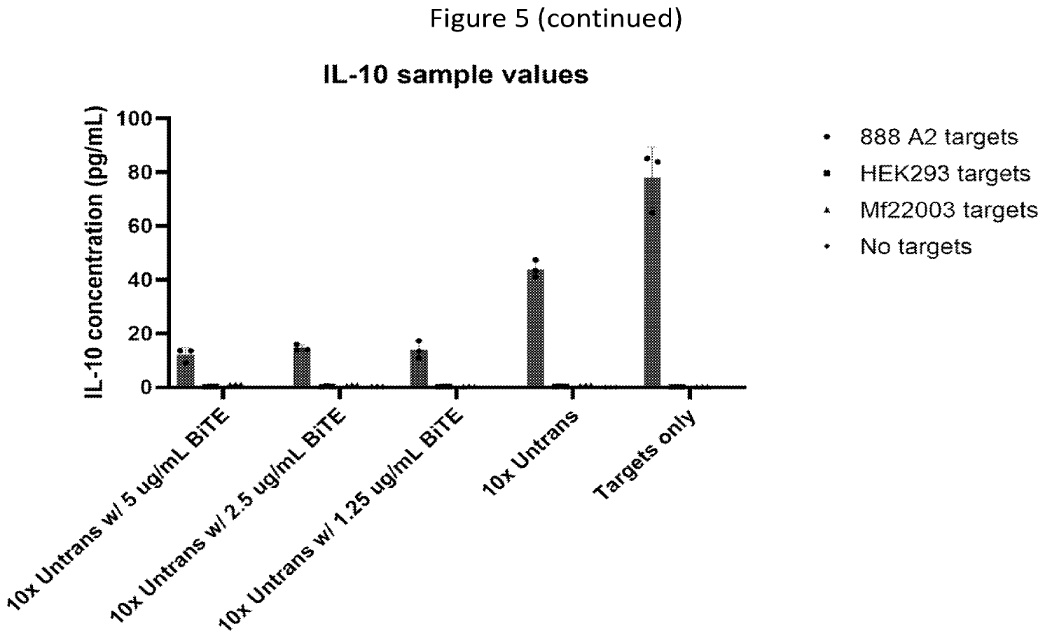

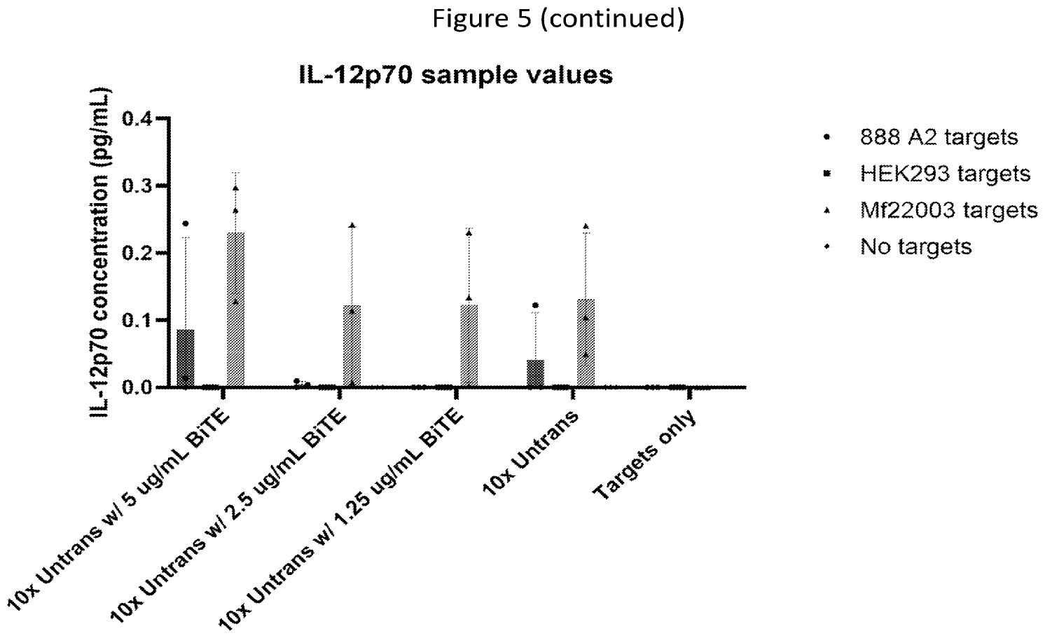

T cell-target cell co-cultures were established to test the ability of the TYRP1 BTE to activate T cells against TYRP1-expressing melanoma cells. Healthy T cells were incubated with human melanoma cells (888 A2), human melanocytes (Mf22003), or negative control cells (HEK293) at a 10:1 effector:target ratio, in presence or absence of the TYRP1 BTE (1.25, 2.5, or g/ml). The ability of the TYRP1 BTE to promote T cell IFN-γ production in the presence of TYRP1-expressing melanoma cells was measured by performing a sandwich ELISA on the co-culture supernatants after 48 hours of incubation. Concentrations of IFN-γ were significantly increased in the presence of the TYRP1 BTE (FIG. 3). Additionally, the ability of the TYRP1 BTE to promote T cell cytotoxicity against TYRP1-expressing melanoma cells was tested by measuring the viability of the co-cultured target cells via an IncuCyte analysis. Melanoma cells showed reduced viability in the presence of the TYRP1 BTE (FIG. 4). Finally, the ability of the TYRP1 BTE to promote T cell production of both pro-inflammatory and anti-inflammatory cytokines in the presence of TYRP1-expressing melanoma cells was tested by performing a bulk multiplex cytokine analysis using the Mesoscale discovery platform. Production of the inflammatory markers IL-10, IL-6, and IL-8 was elevated in the presence of the TYRP1 BTE, though IL-6 production was not elevated in response to melanoma cells (FIG. 5).

Additionally, the ability of the TYRP1 BTE to suppress tumor growth was tested in vivo in a mouse model of melanoma. Mice were subcutaneously injected with 888 A2 melanoma cells, and tumor size was monitored. Mice treated with a combination of the TYRP1 BTE and T cells showed suppression of tumor growth well beyond the treatment dates (FIG. 6A, FIG. 6B).

### Example 2

In the following example, the inventors demonstrate that TYRP1 is expressed in melanoma brain metastases.

### Materials and Methods

### Patient Samples

The tissues of 17 patients with melanoma brain metastases were obtained from the Nervous System Tissue Bank, Northwestern University (IRB approval STU00095863). After deparaffinization and antigen retrieval, tissue sections were blocked with an alkaline blocking reagent (Abcam) and stained with rabbit polyclonal anti-TYRP1 antibody diluted at 1:500 (Santa Cruz Biotech). Bound antibodies were then detected using biotin-conjugated donkey anti-rabbit antibody and streptavidin-peroxidase, each diluted 1:500 (Jackson ImmunoResearch). Warp Red was used as a chromogen (BioCare).

### Quantification of TYRP1 Cell Density in Tumor Samples

After scanning the stained slides, the HistoQuest v.6.0 software (TissueGnostics) was used to determine the TYRP1 cell density in tumor areas. For quantification, a segmentation technique was applied, wherein the nucleus was isolated from the cytoplasm through a ring mask with an interior radius of −0.23 mm and an exterior radius of 0.91 mm, along with a cytoplasm cell mask. This helped define the cytoplasm for the quantification process. For the detection of nuclei, the software's parameters were fine-tuned to identify hematoxylin (with values of 128, 81, 135) and the Warp Red color (with RGB values of 140, 21, and 77) together with hematoxylin. A mean intensity threshold of 23 was established to recognize TYRP1-positive (TYRP1+) cells. In cases where multiple tissues were present in a tumor's slides, they were quantified, and the results were averaged to arrive at a single value for that tumor sample. The TYRP1 cell density was then characterized as the count of TRYP1+ cells in a specified area (mm²).

### Animal Model of Melanoma Brain Metastases

6-8 weeks-old nude mice (Envigo) were used to establish brain metastases. The 888A2+ cell line, which expresses TYRP1, and the A2068 melanoma cell line (obtained from ATCC), which is negative for TYRP1, were modified to express firefly luciferase (ffluc) to enable in vivo imaging of developing metastases. The 888A2+ ffluc cells were injected at 3×10⁶ cells per mouse via an intracarotid artery and monitored for the development of metastases in the brain until the study endpoint using bioluminescent imaging (BLI). At the study endpoint, the brain of mice was harvested and fixed in 10% buffered formalin. Paraffin tissue sections were cut at 4 μm and stained for hematoxylin-eosin stain to visualize the metastatic tissue in the brain.

### Results

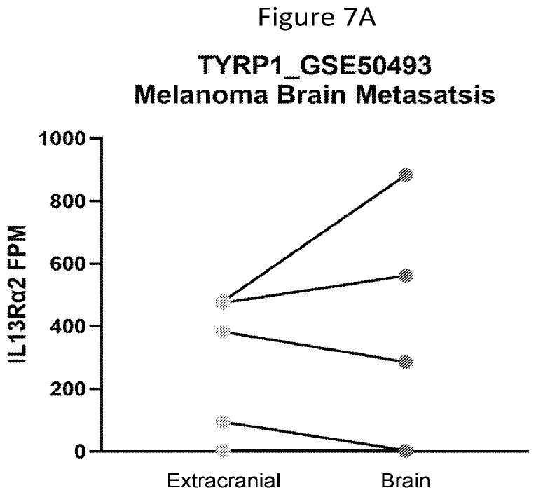

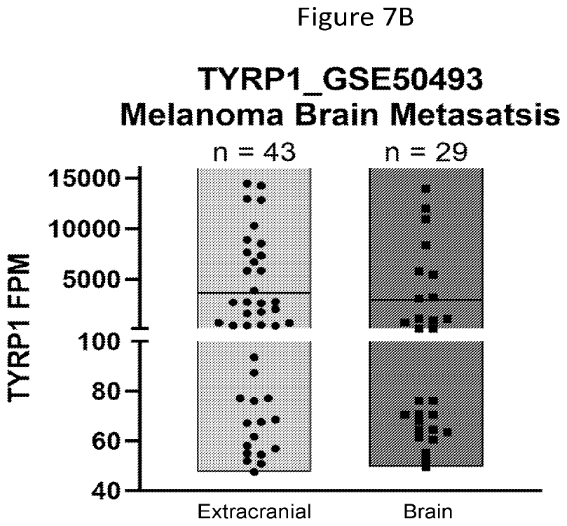

A comparative analysis of TYRP1 expression in extracranial and brain melanoma metastases was performed using an existing GSCE50493 data set (Chen et al., Clin Cancer Res. 20(21):5537-46, 2014). TYRP1 expression in brain melanoma metastases is similar to that in extracranial melanoma metastases in 5 paired samples (i.e., extracranial and intracranial tissue samples derived from the same patient) (FIG. 7A). An analysis of all available tissues from extracranial and brain metastases demonstrates that melanoma metastases found in the peripheral and central nervous systems express similar levels of TYRP1 (FIG. 7B).

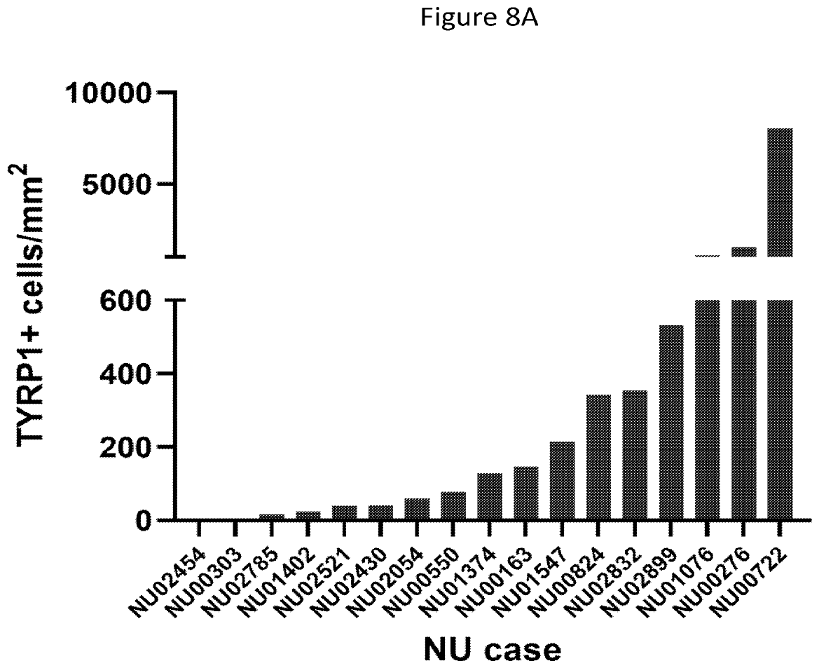

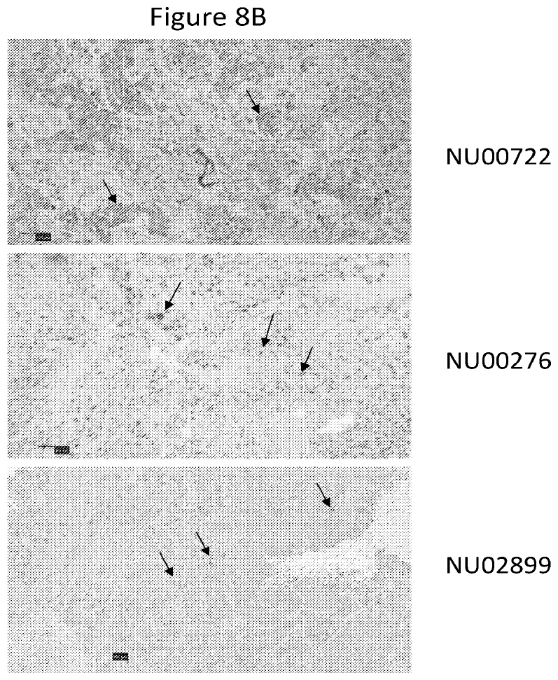

An analysis of TYRP1 expression in melanoma tissue from a data set available from the Human Protein Atlas shows that more than 50-70% of patients express TYRP1 at medium and high levels (data not shown), and a quantitative analysis of melanoma brain metastases obtained through the Northwestern University Nervous System Tumor Bank shows that about 50% of patients express TYRP1 at medium to high levels. An arbitrary expression threshold was set, wherein identification of less than 100 TYRP1-positive cells per mm² was considered low expression. Low expression was observed in 8 out of 17 patients (FIG. 8A, FIG. 8B). These data confirm that melanoma brain metastatic tissue retains the expression of TYRP1 found in peripheral primary and metastatic tissue and validates TYRP1 as a target for treating peripheral melanoma and brain metastases.

Mouse models of TYRP+888A2+ and TYRP− A2068 brain metastatic melanoma were established using the intracarotid delivery route for melanoma cells (data not shown). Both cell lines formed brain metastases in mice, but the A2068 cell line showed faster tumor development. Thus, this cell line will be further modified to express TYRP1 for preclinical testing of TYRP1 BTE activity.

1. A bispecific antibody comprising a first single-chain variable fragment (scFv) that binds to CD3ε and a second scFv that binds to tyrosinase-related protein 1 (TYRP1),
wherein the first scFv comprises:
a) a first heavy chain variable domain (V_{H}) comprising a CDR1 of SEQ ID NO:5, a CDR2 of SEQ ID NO:6, and a CDR3 of SEQ ID NO:7;
b) a first linker; and
c) a first light chain variable domain (V_{L}) comprising a CDR1 of SEQ ID NO:9, a CDR2
having the amino acid sequence DAS, and a CDR3 of SEQ ID NO:10; and
wherein the second scFv comprises:
a) a second V_{L} comprising a CDR1 of SEQ ID NO:13, a CDR2 having the amino acid sequence DAK, and a CDR3 of SEQ ID NO:14;
b) a second linker; and
c) a second V_{H} comprising a CDR1 of SEQ ID NO:16, a CDR2 of SEQ ID NO:17, and a CDR3 of SEQ ID NO:18.

2. The bispecific antibody of claim 1, wherein the bispecific antibody comprises from N-terminus to C-terminus: the first V_{H}, the first linker, the first V_{L}, the second V_{L}, the second linker, and the second V_{H}.

3. The bispecific antibody of claim 1, wherein:
a) the first V_{H} comprises SEQ ID NO:4 or an amino acid sequence with at least 90% sequence similarity to SEQ ID NO:4;
b) the first V_{L} comprises SEQ ID NO:8 or an amino acid sequence with at least 90% sequence similarity to SEQ ID NO:8;
c) the second V_{L} comprises SEQ ID NO:12 or an amino acid with at least 90% sequence similarity to SEQ ID NO:12; and/or
d) the second V_{H} comprises SEQ ID NO:15 or an amino acid with at least 90% sequence similarity to SEQ ID NO:15.

4. The bispecific antibody of claim 3, wherein:
a) the first scFv comprises SEQ ID NO:3 or an amino acid sequence with at least 90% sequence similarity to SEQ ID NO:3; and/or
b) the second scFv comprises SEQ ID NO:11 or an amino acid sequence with at least 90% sequence similarity to SEQ ID NO:11.

5. (canceled)

6. The bispecific antibody of claim 1, wherein the first linker and the second linker are (Gly4S)₃ (SEQ ID NO:19).

7. The bispecific antibody of claim 1, wherein the first scFv and the second scFv are linked via a third linker.

8. (canceled)

9. The bispecific antibody of claim 82, wherein the third linker is SEQ ID NO:20.

10. The bispecific antibody of claim 1, further comprising a signal peptide on the 5′ end.

11. The bispecific antibody of claim 10, wherein the signal peptide is SEQ ID NO:21.

12. The bispecific antibody of claim 1, further comprising a tag.

13. The bispecific antibody of claim 1, wherein the bispecific antibody comprises SEQ ID NO:2.

14-17. (canceled)

18. A pharmaceutical composition comprising the bispecific antibody of claim 1 and a pharmaceutically acceptable carrier.

19. A nucleic acid encoding the bispecific antibody of claim 1.

20. (canceled)

21. A transgenic cell that expresses the bispecific antibody of claim 1.

22. A method of treating a TYRP1-expressing tumor in a subject, the method comprising: administering a therapeutically effective amount of the bispecific antibody of claim 1 to the subject to treat the tumor.

23. The method of claim 22, further comprising determining whether TYRP1 is expressed on a tumor cell in the subject, wherein the bispecific antibody is only administered to the subject if TYRP1 expression is detected.

24. The method of claim 22, wherein the tumor cell is melanoma.

25. A method for inducing lysis of a TYRP1-expressing target cell, the method comprising: contacting the target cell with the bispecific antibody of claim 1 in the presence of a T cell in an amount effective to lyse the target cell.

26. The method of claim 25, wherein the T cell is a cytotoxic T cell.

27. The method of claim 25, wherein the contacting is performed in vivo in a subject that has a tumor.


SEQUENCE LISTING (26 sequences)

SEQ ID NO: 1 - DNA, 1602 bp, synthetic construct
  source 1..1602: mol_type = other DNA
atgggatggtcctgtatcatcctgttcctggtggccaccgccaccggcgtgcacagccaggtgcaactggtagagtctggcggaggcgttgtgcagcccggacgcagcctgcggctgagctgcgccgccagcggcttcaagttcagcgggtacggaatgcactgggtgcggcaggctcctggcaagggcctggaatgggtggctgtgatctggtacgacgsctccaaaaagtactacgtggacagcgtgaaaggcagattcacaattagcagagataacagcaagaacaccctgtacctgcagatgaatagcctgagagctgaggatacagccgtgtactactgcgccagacagatgggctactggcacttcgacctgtggggaaggggcacccttgttaccgtgagctccggaggaggcggctctggaggcggcggctcaggcggaggcggctccgagatcgtgctgacacagagccctgccaccctgagcctgtctcccggcgaaagagccacactgagctgtagagccagccagagcgtgagcagctacctggcctggtaccagcaaaaacccggtcaggcccctagactgctaatctacgacgcctctaatagagctacaggcatccccgctagatttagcggaagcggctccggcaccgacttcaccctgaccatctcttctctggaacctgaggattttgccgtgtactattgccagcagagaagtaactggcctccactgaccttcggcggagggaccaaggtggaaatcaagggcggaggctctggcggcggcggaagcggtagcggaggcggcagcggcggcggcggctctggtggcggcgatatccagatgagccagagccctgcctctctgagcgcctccgtgggcgagacagtgaccatcacctgtagagccagcggaaatatctacaactacctggcttggtatcagcagaaacagggcaagagcccccacctgctggtctacgacgccaagaccctggccgacggcgtgccatctagattcagcggctctggaagcggcacccagtacagcctgaagatcagcagcctccagaccgaggacagcggcaactactactgccaacacttctggtccctgccttttacattcggctccggcacaaagctggaaatcaagcggggcggcggaggcagcggcggcggaggcagcggcgggggcggcagcgaagtgcagctgcagcagagcggagccgagctggtgcggcctggcgccctggtcaagctgagctgcaagaccagcggcttcaacatcaaggactacttcctgcactgggtgcggcagagacctgatcagggcctggaatggatcggctggatcaaccccgacaacgscaacaccgtgtacgacccaaagttccagggaacagcttctctgaccgctgatacaagctctaataccgtgtacctgcaactctccggcctgacaagcgaggacaccgccgtgtatttttgtaccagaagagattacacctacgagaaagccgccctggactactggggccagggcaccaccgttacagtgtccagccaccaccaccaccaccattga

SEQ ID NO: 2 - protein, 533 aa, synthetic construct
  VARIANT 74: note = X is G or A
  VARIANT 462: note = X is G or A
MGWSCIILFLVATATGVHSQVQLVESGGGVVQPGRSLRLSCAASGFKFSGYGMHWVRQAPGKGLEWVAVIWYDXSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGSGGGGSGSGGGSGGGGSGGGDIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKRGGGGSGGGGSGGGGSEVQLQQSGAELVRPGALVKLSCKTSGFNIKDYFLHWVRQRPDQGLEWIGWINPDNXNTVYDPKFQGTASLTADTSSNTVYLQLSGLTSEDTAVYFCTRRDYTYEKAALDYWGQGTTVTVSSHHHHHH

SEQ ID NO: 3 - protein, 240 aa, synthetic construct
  VARIANT 55: note = X is G or A
QVQLVESGGGVVQPGRSLRLSCAASGFKFSGYGMHWVRQAPGKGLEWVAVIWYDXSKKYYVDSVKGRFISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 4 - protein, 117 aa, synthetic construct
  VARIANT 55: note = X is G or A
QVQLVESGGGVVQPGRSLRLSCAASGFKFSGYGMHWVRQAPGKGLEWVAVIWYDXSKKYYVDSVKGRFISRDNSKNTLYLQMNSLRAEDTAVYYCARQMGYWHFDLWGRGTLVTVSS

SEQ ID NO: 5 - protein, 8 aa, synthetic construct
GFKFSGYG

SEQ ID NO: 6 - protein, 8 aa, synthetic construct
  VARIANT 5: note = X is G or A
IWYDXSKK

SEQ ID NO: 7 - protein, 11 aa, synthetic construct
ARQMGYWHFDL

SEQ ID NO: 8 - protein, 108 aa, synthetic construct [sequence omitted]

SEQ ID NO: 9 - protein, 6 aa, synthetic construct
QSVSSY

SEQ ID NO: 10 - protein, 10 aa, synthetic construct
QQRSNWPPLT

SEQ ID NO: 11 - protein, 244 aa, synthetic construct
  VARIANT 179: note = X is G or A
DIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKRGGGGSGGGGSGGGGSEVQLQQSGAELVRPGALVKLSCKTSGFNIKDYFLHWVRQRPDQGLEWIGWINPDNXNTVYDPKFQGTASLTADTSSNTVYLQLSGLTSEDTAVYFCTRRDYTYEKAALDYWGQGTTVTVSS

SEQ ID NO: 12 - protein, 108 aa, synthetic construct
DIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKR

SEQ ID NO: 13 - protein, 6 aa, synthetic construct
GNIYNY

SEQ ID NO: 14 - protein, 9 aa, synthetic construct
QHFWSLPFT

SEQ ID NO: 15 - protein, 121 aa, synthetic construct
  VARIANT 56: note = X is G or A
EVQLQQSGAELVRPGALVKLSCKTSGFNIKDYFLHWVRQRPDQGLEWIGWINPDNXNTVYDPKFQGTASLTADTSSNTVYLQLSGLTSEDTAVYFCTRRDYTYEKAALDYWGQGTTVTVSS

SEQ ID NO: 16 - protein, 8 aa, synthetic construct
GFNIKDYF

SEQ ID NO: 17 - protein, 8 aa, synthetic construct
  VARIANT 6: note = X is G or A
INPDNXNT

SEQ ID NO: 18 - protein, 14 aa, synthetic construct
TRRDYTYEKAALDY

SEQ ID NO: 19 - protein, 15 aa, synthetic construct
GGGGSGGGGSGGGGS

SEQ ID NO: 20 - protein, 23 aa, synthetic construct
GGGSGGGGSGSGGGSGGGGSGGG

SEQ ID NO: 21 - protein, 19 aa, synthetic construct
MGWSCIILFLVATATGVHS

SEQ ID NO: 22 - protein, 6 aa, synthetic construct
HHHHHH

SEQ ID NO: 23 - protein, 8 aa, synthetic construct
IWYDGSKK

SEQ ID NO: 24 - protein, 8 aa, synthetic construct
IWYDASKK

SEQ ID NO: 25 - protein, 8 aa, synthetic construct
INPDNGNT

SEQ ID NO: 26 - protein, 8 aa, synthetic construct
INPDNANT